SEQUENCE LISTING (14 sequences)

SEQ ID NO: 1 - protein, 250 aa, synthetic construct
MGSSMSKGEELFTGVVPILVELDGDVNGHKFSVRGEGEGDATIGKLTLKFICTTGKLPVPWPTLVTTLTYGVQCFSRYPDHMKRHDFFKSAMPEGYVQERTISFKDDGKYKTRAVVKFEGDTLVNRIELKGTDFKEDGNILGHKLEYNFNSHNVYITADKQKNGIKANFTVRHNVEDGSVQLADHYQQNTPIGDGPVLLPDNHYLSTQTVLSKDPNEKRDHMVLLEFVTAAGITHGMDELYKLEHHHHHH

SEQ ID NO: 2 - protein, 172 aa, synthetic construct
MSKGEELFTGVVPILVELDGDVNGHKFSVRGEGEGDATIGKLTLKFICTTGKLPVPWPTLVTTLTYGVQCFSRYPDHMKRHDFFKSAMPEGYVQERTISFKDDGKYKTRAVVKFEGDTLVNRIELKGTDFKEDGNILGHKLEYNFNSHNVYITADKGTCFNGGENLYFQGAS

SEQ ID NO: 3 - protein, 93 aa, synthetic construct
QKNGIKANFTVRHNVEDGSVQLADHYQQNTPIGDGPVLLPDNHYLSTQTVLSKDPNEKRDHMVLLEFVTAAGITHGMDELYKTSLPETGGGEL

SEQ ID NO: 4 - protein, 167 aa, synthetic construct
MISLIAALAVDRVIGMENAMPWNLPADLAWFKRNTLNKPVIMGRHTWESIGRPLPGRKNIILSSQPGTDDRVTWVKSVDEAIAACGDVPEIMVIGGGRVYEQFLPKAQKLYLTHIDAEVEGDTHFPDYEPDDWESVFSEFHDADAQNSHSYCFEILERRLEHHHHHH

SEQ ID NO: 5 - protein, 106 aa, synthetic construct
CFNGGENLYFQGASLPADLAWFKRNTLNKPVIMGRHTWESIGRPLPGRKNIILSSQPGTDDRVTWVKSVDEAIAACGDVGGMISLIAALAVDRVIGMENAMPWNGT

SEQ ID NO: 6 - protein, 91 aa, synthetic construct
CFNGGHHHHHHELPEIMVIGGGRVYEQFLPKAQKLYLTHIDAEVEGDTHFPDYEPDDWESVFSEFHDADAQNSHSYCFEILERRGGSGGTS

SEQ ID NO: 7 - protein, 113 aa, synthetic construct
MGSSAHIVMVDAYKPTKGEDSATHIKFSKRDEDGKELAGATMELRDSGKTISTWISDGQVKDFYLYPGKYTFVETAAPDGYEVATAITFTVNEQGQVTVNGKATKLEHHHHHH

SEQ ID NO: 8 - protein, 74 aa, synthetic construct
GGGELGEDSATHIKFSKRDEDGKELAGATMELRDRSGGSGGSENLYFQGGSGGSAHIVMVDAYKPTKGGSGGSG

SEQ ID NO: 9 - protein, 81 aa, synthetic construct
CFNASHHHHHHSGKTISTWISDGQVKDFYLYPGKYTFVETAAPDGYEVATAITFTVNEQGQVTVNGKATKGGSGGSGGSGT

SEQ ID NO: 10 - protein, 74 aa, synthetic construct
GGGELGEDSATHIKFSKRDEDGKELAGATMELRDRSGGSGGSENLYFQGGSGGSAHIVMVAAYKPTKGGSGGSG

SEQ ID NO: 11 - protein, 155 aa, synthetic construct
MKGSSHHHHHHVEASSGKTISTWISDGQVKDFYLYPGKYTFVETAAPDGYEVATAITFTVNEQGQVTVNGKATKGGSGGSGGSASGEDSATHIKFSKRDEDGKELAGATMELRDRSGGSGGSENLYFQGGSGGSAHIVMVDAYKPTKGGSGGSGG

SEQ ID NO: 12 - protein, 155 aa, synthetic construct
MKGSSHHHHHHVEASSGKTISTWISDGQVKDFYLYPGKYTFVETAAPDGYEVATAITFTVNEQGQVTVNGKATKGGSGGSGGSASGEDSATHIKFSKRDEDGKELAGATMELRDRSGGSGGSENLYFQGGSGGSAHIVMVAAYKPTKGGSGGSGG

SEQ ID NO: 13 - protein, 155 aa, synthetic construct
MKSHHHHHHKLGGSGGSENLYFQGGTGSGGSGEDSATHIKFSKRDEDGKELAGATMELRDGSASSGKTISTWISDGQVKDFYLYPGKYTFVETAAPDGYEVATAITFTVNEQGQVTVNGKATKGGSGGSGGSGSAHIVMVDAYKPTKGGSGGSGG

SEQ ID NO: 14 - protein, 155 aa, synthetic construct
MKSHHHHHHKLGGSGGSENLYFQGGTGSGGSGEDSATHIKFSKRDEDGKELAGATMELRDGSASSGKTISTWISDGQVKDFYLYPGKYTFVETAAPDGYEVATAITFTVNEQGQVTVNGKATKGGSGGSGGSGSAHIVMVAAYKPTKGGSGGSGG

(19) United States
(12) Patent Application Publication
(10) Pub. No.: US 2025/0378902 A1
(43) Pub. Date: Dec. 11, 2025

(54) PROGRAMMATIC DESIGN METHOD FOR TOPOLOGICAL PROTEIN

(71) Applicant: Peking University, Beijing (CN)

(72) Inventors: Wenbin Zhang, Beijing (CN); Wenhao WU, Beijing (CN); Yuxiang WANG, Beijing (CN); Jing FANG, Beijing (CN); Zhiyu QU, Beijing (CN)

(21) Appl. No.: 19/231,304

(22) Filed: Jun. 6, 2025

Related U.S. Application Data
(63) Continuation of application No. PCT/CN2023/136513, filed on Dec. 5, 2023.

(30) Foreign Application Priority Data
Dec. 7, 2022 (CN) 202211565469.0

(51) Int. Cl. G16B 15/20; G16B 15/30; G16B 30/20; G16B 40/20

(52) U.S. Cl. CPC G16B 15/20; G16B 15/30; G16B 30/20; G16B 40/20

(57) ABSTRACT

The present disclosure discloses a programmatic design method for a topological protein, the method comprising the following steps: i) splitting an original structure of a protein-of-interest, and designing possible rewiring approaches according to a target topological structure; ii) evaluating connection approaches between structural motifs and determining priorities of the connection approaches in a subsequent design; iii) for each of the connection approaches, generating new virtual loop regions successively, exhausting all possible combinations of generation orders of the loop regions, creating corresponding spatial relationships, determining the formed chemical topological structures, calculating a formation probability of the target topological structure, and determining a length range of a newly-generated loop region; and iv) designing a length and sequence of the newly-generated loop region of the topological protein. The programmatic design method for topological proteins provided in the present disclosure provides a desirable platform for illustrating the structure-activity relationship of topological proteins and also provides a convenient method for developing functional topological proteins.

### CROSS-REFERENCE TO RELATED APPLICATION

This application is a continuation of International Application No. PCT/CN2023/136513, filed on May 12, 2023, which claims priority to CN application Ser. No. 20/221,1565469.0, filed on Dec. 7, 2022, the contents of each of which are herein incorporated by reference in the entirety.

### SEQUENCE LISTING

The instant application contains a Sequence Listing which has been submitted in XML format via Patent Center and is hereby incorporated by reference in its entirety. Said XML copy, created Apr. 24, 2025, is named 6984-2163173US.xml and is 14,921 bytes in size.

### TECHNICAL FIELD

The present disclosure relates to the field of the design and synthesis of topological proteins, more specifically, to a programmatic design method for topological proteins.

### BACKGROUND

Topological proteins are a class of nonlinear proteins with complex chemical topology, which provide a brand-new subject of research for the fields such as antibody engineering, industrial enzymes, and biomaterials, and have important fundamental scientific significance and application value. Dictated by the central dogma of life, intracellular protein synthesis follows a rigorous template synthesis mechanism, which makes it difficult to synthesize structures beyond a linear backbone structure. Biosynthesis of natural topological proteins often involves complex post-translational modification processes, and many mechanisms remain unknown, so it can hardly be applied to the design and synthesis of artificial topological proteins. However, inspired by how nature makes topological proteins, proteins with complex backbone chemical topologies can be derived from linear protein precursors by controlling the spatial relationship of protein chains in conjunction with efficient and specific chemical reactions of proteins.

People have currently developed some strategies for the synthesis of topological proteins based on the “assembly-reaction” synergy, in which known genetically encoded protein entangling motifs and reaction motifs are used to realize the biosynthesis of proteins with specific topological structures (such as knots and links). However, topology engineering of proteins is still in the initial stage as a whole. On the one hand, this is because protein entangling motifs as templates are very limited and are difficult to discover and develop. On the other hand, this is because the size of the protein entangling motifs and reaction motifs is relatively big, such that there are redundant residual motifs left in the final topological protein construct. It not only affects the illustration of the structure-activity relationship of the topological proteins, but also fails to take the most advantages of the stabilization effect of the chemical topology, making the problem rather complicated. It is an urgent need to get rid of the limitation of these templates, so as to redesign the currently known proteins with a linear backbone into topological proteins with a special backbone topology in a “traceless” manner. By doing so, one can give full play to the advantages of chemical topology in reshaping the proteins' conformational space.

In view of the foregoing, the present disclosure is committed to introducing entanglement into the protein-of-interest by editing the connectivity of the protein-of-interest to realize the “trace-less” or “micro-trace” topological transformation strategy in the hope of developing a systematically and universally programmatic design method for topological proteins.

### SUMMARY

### Technical Problem

In view of the problems existing in the prior art, such as few variety and large size of the protein entangling motifs and reaction motifs, the present disclosure provides a programmatic design method for converting a linear protein into a topological protein by editing the connectivity and spatial relationship between the secondary structures of the protein-of-interest itself.

### Solution to Problem

In view of the problems existing in the prior art, the present inventors have conducted extensive researches and repeated experiments, and thus completed the present disclosure by designing a topological protein variant with similar tertiary structure and sequence composition but different backbone chemical topology as compared to the protein-of-interest through introducing artificial entanglement within or between the protein chains by changing the connectivity between the secondary structural motifs while retaining the tertiary structure of the protein-of-interest. Specifically, the present disclosure is as follows:

In the first aspect, the present disclosure provides a programmatic design method for a topological protein, the method comprising the following steps:
i) splitting an original structure of a protein-of-interest, and designing possible rewiring approaches according to a target topological structure;
ii) evaluating connection approaches between structural motifs and determining priorities of the connection approaches in a subsequent design:
iii) for each of the connection approaches, generating new virtual loop regions successively, exhausting all possible combinations of generation orders of the loop regions, creating corresponding spatial relationships, determining the chemical topology of the formed structures, calculating a formation probability of the target topological structure, and determining a length range of a newly-generated loop region: and
iv) designing a length and sequence of the newly-generated loop region of the topological protein.

In some specific embodiments, the specific operations of the steps i) to iv) in the above-mentioned programmatic design method for the topological protein are as follows:
i) splitting an original tertiary structure of the protein-of-interest by using a secondary structure as a motif, successively increasing the number of the loop regions to be split starting from two loop regions, giving priority to an approach where fewer loop regions will be split, determining a splitting approach, and then designing all possible new connection approaches between secondary structural motifs of the protein based on the target chemical topological structure:
ii) scoring and evaluating the new connection approaches designed in step i), and determining the priorities of different connection approaches in the subsequent design of a topological structure based on the results of scoring and evaluation:
iii) further designing the spatial relationship of each of the connection approaches in order of priorities based on the results of the scoring and evaluation in step ii), generating the corresponding spatial relationship by successively generating the new virtual loop region at each of positions to be connected and exhausting all possible combinations of the generation orders of the loop regions, determining the chemical topological structures of the protein obtained after generating the new virtual loop regions, and calculating the formation probability of the target topological structure in said connection approach, thereby determining a relative spatial relationship and the length range of the virtual loop region corresponding to the formation of the target topological structure in said connection approach: and
iv) preferably selecting specific lengths of actual loop regions based on the relative spatial relationship and the length range of the virtual loop region determined in step iii), and further designing the amino acid sequences of the actual loop regions as the sequences of the newly-generated loop regions of the topological protein to obtain a final topological protein.

In a specific embodiment, the splitting according to step i) is the splitting carried out at the loop region: and the connection according to step i) means generation of a new loop region between the N-terminus and the C-terminus of the secondary structural motifs that are obtained after splitting.

According to the programmatic design method for the topological protein provided in the first aspect of the present disclosure, the original tertiary structure of the protein-of-interest in step i) contains N secondary structural motifs and N loop regions, wherein the loop regions comprise the virtual loop region between the original N-terminus and C-terminus, and when the topological structure of a designed protein-of-interest is [2] catenane, the original tertiary structure of the protein-of-interest is split by the following method and a new connection approach is determined:
(1) splitting two loop regions in the original tertiary structure of the protein-of-interest, with a total of N(N-1)/2 splitting approaches and N(N-1)/2 new connection approaches:
(2) splitting three loop regions in the original tertiary structure of the protein-of-interest, with a total of N(N-1)(N-2)/6 splitting approaches and N(N-1)(N-2)/2 new connection approaches: or
(3) splitting M loop regions in the original tertiary structure of the protein-of-interest, with a total of N!/[(N-M)!×M!] splitting approaches and

$$\frac{N !}{( N - M ) ! \times M !} \times {\sum }_{L = 1}^{M - 1} ( M ! / 2 L ( M - L ) )$$

new connection approaches, wherein M is 4 or an integer greater than 4 and wherein L is a positive integer from 1 to M-1.

In some preferred embodiments, subsequent evaluations and designs are performed successively in order from the smallest to the largest number of the loop regions required to be rewired.

According to the design method for the topological protein provided in the first aspect of the present disclosure, a basis for the evaluation in step ii) is as follows:
setting two evaluation criteria for each of the positions to be connected, i.e., (a) a Euclidean distance between the structural motifs to be connected, and (b) a probability that the loop regions generated between the to-be-connected secondary structural motifs conform to the statistical law of the loop regions of all natural proteins:
calculating the probability of generating the new loop regions at the positions to be connected based on the above evaluation criteria (a) and (b); and
calculating an overall generation probability taking account of all loop regions required to be regenerated in a current connection approach, scoring and ranking all connection approaches based on the probability, and selecting superior scoring groups for subsequent successive design.

In a specific embodiment, the basic principles of the scoring are as follows:
(a1) counting the Euclidean distances of all loop regions in Protein Data Bank (PDB), calculating a ratio of the number of the loop regions corresponding to each of the Euclidean distances to the total number of the loop regions, and taking the ratio as the probability p1 of generating the loop regions at said Euclidean distance:
(b1) counting the Euclidean distance between the loop regions and the lengths of the loop regions of all proteins in PDB to obtain probability distribution of the lengths of the loop regions at a specific Euclidean distance, taking the virtual loop region generated at a target position by a minimum solvent accessible path as a minimum length of the loop region that is actually generated, taking the minimum length as a lower limit of integration, taking the longest length of the loop region available counted at the current Euclidean distance as an upper limit of the integration, performing an integral calculation on the probability distribution to obtain the probability p2 that the actually generated loop regions conform to the statistical law:
(c1) taking a product of the probabilities calculated in (a1) and (b1) as the probability p of actually generating the loop regions at the current position, i.e., p=p1×p2; and
(d1) taking a product of the probabilities of generating the loop regions calculated at all positions to be connected in the current connection approach as the probability pₜₒₜₐₗ, i.e., Pₜₒₜₐₗ=Πpᵢ, of said connection approach, wherein said i is the number of all new loop regions required to be generated, performing the scoring and ranking based on the probability, and determining the priorities in the subsequent designs of the topological structures based on the ranking.

According to the design method for the topological protein provided in the first aspect of the present disclosure, the step iii) comprises: generating new virtual loop regions between the secondary structural motifs by a minimum solvent accessible path in specific connection approaches: generating more than one new virtual loop region in each of the connection approaches, and exhausting all possible spatial relationships between newly-generated virtual loop regions by exhausting all combinations of the generation orders of the virtual loop regions, wherein the total number of all possible spatial relationships is set to n:
determining the topological structure of the designed protein corresponding to the newly-generated virtual loop regions in each of the generation order by calculating the Gauss linking number or the knot invariant, to obtain the number m of the topological structures that match the target topological structure:
calculating the probability m/n of generating the target topological structure in the current connection approach based on said n and m, and outputting the probability as the formation probability of the target topological structure in a current connection relationship: and
determining the length ranges of the actual loop regions (for example, taking the length of the correspondingly generated virtual loop region, i.e., the number l of the virtual points, as the lower limit and taking l+30 as the upper limit) based on the connectivity and the spatial relationship that enable formation of the target topological structure, and imposing a length limitation based on the relative spatial relationships of the loop regions, and defining the length range of each of the loop regions that are adjacent to and cross each other based on the requirement that the difference between the length of the loop region relatively far away from the hydrophobic core of the folded protein and the length of the loop region relatively close to the hydrophobic core of the folded protein is not less than the length difference in the lower limits thereof, in order to maintain the relatively spatial relationships unchanged, thereby outputting the design result that matches the target topological structure and the length range of the corresponding newly-generated actual loop region.

According to the design method for the topological protein provided in the first aspect of the present disclosure, the step iv) comprises: selecting, based on the length ranges of the actual loop regions determined in step iii), combinations of the lengths of the loop regions that are more aligned with the basis for scoring and evaluation in step ii) as the specific number of the amino acids in each of the actual loop regions, and designing amino acid sequences of the actual loop regions:
preferably, the amino acid sequences of the actual loop regions are designed by any one of the following three methods: (a) directly designing a flexible linking loop region with the target number of amino acids, wherein the flexible linking loop region comprises any one of an enzyme cleavage site, an affinity purification tag, a residual motif after a coupling reaction, part or full sequence of the original loop region, or linking sequences consisting of glycine G and serine S, or any combination thereof: (b) searching structures similar to the two termini of the motifs to be connected in PDB by a similar structural motif search algorithm, and selecting the lengths of the loop regions that meet the requirement as the sequences of the loop regions: and (c) designing the sequences of linking loop regions with target lengths by a computer-assisted means:
more preferably, the enzyme cleavage site is any one of a Tobacco Etch Virus protease cleavage site (ENLYFQG), a Tobacco Vein Mottling Virus protease cleavage site (ETVRFQG), an enterokinase cleavage site (DDDDK), a coagulation factor Xa protease cleavage site (IDGR) or a WELQut protease cleavage site (WELQ): the affinity purification tag is any one of Histag (HHHHHH), Strep-Tag II (WSHPQFEK) or a Flag tag (DYKDDDDK); the residual amino acid sequence after the coupling reaction is any one of CFN, ESGSGK, LPETG or NHV; the similar structural motif search algorithm is any one of MASTER, FragBag or TOPOFIT: and the computer-assisted means is any one of a Rosetta loop modelling method, a SCUBA method, or a FoldX LoopReconstruction method.

According to the design method for the topological protein provided in the first aspect of the present disclosure, the chemical topological structure of the topological protein is any one selected from the group consisting of a branched structure, a multicyclic structure, a knot structure, and a link structure, or any combination thereof: preferably, the topological protein is a protein catenane having two or more mechanically-interlocked cyclic structures or a knot protein having a trefoil knot, 4₁ knot, 5₁ knot or 5₂ knot structure.

In the second aspect, the present disclosure provides a topological protein, wherein the topological protein is designed by the method according to the first aspect of the present disclosure.

In some specific embodiments, the chemical topological structure of the topological protein is any one selected from the group consisting of a branched structure, a multicyclic structure, a knot structure, and a link structure, or any combination thereof: preferably, the topological protein is a protein catenane having two or more mechanically-interlocked cyclic structures or a knot protein having a trefoil knot, 4₁ knot, 5₁ knot or 5₂ knot structure.

The technical solutions provided in the first and second aspects of the present disclosure will be further explained and illustrated below.

The tertiary structure of a protein is composed of secondary structural motifs (such as α-helices and β-sheets) and flexible loop regions connecting the secondary structural motifs. It has a relatively conservative structure, in which the loop regions are relatively exposed and highly engineerable. Rewiring the structural motifs by modifying the loop regions can change the chemical topology of the protein backbone without drastically altering the hydrophobic core. On the precondition that the structural motifs of the protein-of-interest remain basically unchanged, the present disclosure designs a variety of rewiring approaches of new loop regions by computer-assisted means, thereby transforming the protein-of-interest into a plurality of variants having specific chemical topological structures.

The technical solutions of the present disclosure will be described in detail below.

The whole design process of the programmatic design method for topological proteins provided in the present disclosure includes the following four main steps and can be fully programmed.

### 1) Rationally Splitting the Original Tertiary Structure of the Protein-of-Interest, and Designing All Possible Rewiring Approaches Between the Secondary Structural Motifs Based on the Target Chemical Topological Structure

There are numerous possibilities for rewiring the secondary structural motifs of the protein-of-interest in three-dimensional space. Since the backbones of nascent proteins synthesized in cells in situ are all linear, assuming a virtual connection between the N-terminus and the C-terminus, on this basis, the whole protein can be divided into N secondary structural motifs and N loop regions. Possible connection approaches for rewiring the secondary structural motifs to obtain single-ring knots (cyclic molecules including unknots) are (N-1)! (i.e. the factorial of N-1). There are more possible connection approaches for rewiring the secondary structural motifs to obtain multi-ring links. For example, the possible connection approaches for realizing two-component links are

$${\sum }_{L = 1}^{N - 1} N ! / 2 L ( N - L ) ,$$

where L is a positive integer from 1 to N-1. These connection approaches may also lead to different chemical topological structures as a result of the difference in the relative spatial relationships between the loop regions and the secondary structural motifs. Considering the design of the sequence of each loop region, the protein sequences and constructions possibly formed eventually are even countless, while only a small fraction of the connection approaches could actually meet the requirements for forming the target topological structure. If we take into further consideration the structural instability and folding kinetic barriers possibly resulting from an alternation of the connection approaches, and problems potentially arising in the subsequent practical synthesis and preparation (e.g., poor assembly-reaction synergy in the synthesis process and various side reactions), only a very small portion of the topology constructions is relatively feasible.

Therefore, instead of exhausting all possible splitting and connection approaches, the present disclosure chooses to engineer as few loop regions as possible based on the chemical topological structure of the protein-of-interest.

As shown in FIG. 1, taking the design of a protein [2] catenane (i.e., a protein catenane containing two mechanically interlocked rings, also referred to as a two-component link) as an example, only two loop regions in the original structure of the protein may be selected for splitting, and the N-terminus and the C-terminus of the resulting two polypeptide chains are cyclized respectively to obtain a two-component link structure. Theoretically, there are a total of N(N-1)/2 splitting approaches, and each splitting approach has one and only one new connection approach, so there are a total of N(N-1)/2 different new connection approaches ultimately. If three loop regions in the original structure of the protein are split, there are a total of N(N-1) (N-2)/6 splitting approaches and there are three approaches for rewiring the loop regions to form a two-component link after each splitting, so there are a total of N(N-1)(N-2)/2 connection approaches, and so forth. Based on these connection approaches, it is possible to obtain protein [2] catenane structures by further designing the spatial relationships. Certainly, it is also possible to split more loop regions as appropriate. For example, if M loop regions in the original structure of the protein are split, there are a total of N!/[(N-M)!×M!] splitting approaches and

$$\frac{N !}{( N - M ) ! \times M !} \times {\sum }_{L = 1}^{M - 1} ( M ! / 2 L ( M - L ) )$$

connection approaches, where M is 4 or an integer greater than 4, and L is a positive integer from 1 to M-1.

The smaller the number of engineered loop regions, the smaller the disturbance to the folded structure, the higher the probability of successfully synthesizing topological proteins eventually. Sequential design in order of the number of the loop regions to be rewired from the least to the most can ensure preferential design of the systems with a high success rate.

### 2) Scoring and Evaluating the Possible Rewiring Approaches Between the Secondary Structural Motifs and Determining the Priorities

By reasonably optimizing the approaches of splitting the loop regions, the possible rewiring approaches are greatly reduced. Thereafter, each of the connection approaches is scored and evaluated, and the system with a high score is selected first for the next step of design, followed by the other systems with low scores. The basis for scoring and evaluation includes the distance between the termini of the secondary structural motifs to be connected and the probability that the generated loop regions conform to the statistical law of the loop regions of the natural proteins.

As shown in (A) of FIG. 2, the Euclidean distances of all loop regions in PDB (Protein Data Bank) are counted, and the ratio of the number of the loop regions corresponding to each of the Euclidean distances to the total number of the loop regions is calculated and taken as the probability p1 that the loop regions can be generated at this Euclidean distance.

As shown in (B) of FIG. 2, the Euclidean distances between the loop regions and the lengths of the loop regions of all proteins in PDB are counted to obtain the probability distribution of the lengths of the loop regions at a specific Euclidean distance. In the course of design, a virtual loop region is generated using a program at a target position by a minimum solvent accessible path (where the “solvent accessible path” refers to a path generated between two points on the surface of the protein that does not collide with the protein, and the shortest path is the “minimum solvent accessible path”, whereby another path is not allowed to cross between this generated path and the protein surface, see Bioinformatics, 2019, 35, 3169-3170) as the minimum length of the loop region that is actually generated. This length is taken as the lower limit of the integration and the longest length of loop region available counted at the current Euclidean distance as the upper limit of the integration to perform an integral calculation on the above probability distribution, to obtain the probability p2 that the loop regions actually generated conform to the statistical law. The solvent accessible distance is specifically calculated by the following method: taking the midpoint of the amino acids to be connected as a center to mesh the surrounding space with a radius of 5 nm, where each mesh is 0.1 nm in size: performing random walks in the space unoccupied by the proteins in the meshes: exhausting all possible paths connecting two amino acids: and selecting the shortest path therefrom as the minimum solvent accessible path.

The product of the probabilities calculated based on the above two influencing factors (the probability p1 and the probability p2 as described above) is taken as the probability p (i.e., p=p1×p2) of the loop regions that can be actually generated at the current position.

Finally, the product of the probabilities for generating the loop regions as calculated at all positions to be connected in the current connection approach is taken as the probability pₜₒₜₐₗ, i.e., pₜₒₜₐₗ=Πpᵢ, of this connection approach, wherein said i is the number of all new loop regions to be generated: the connection approaches are scored and ranked based on the probability: and the priorities in the subsequent designs of the topological structures are determined based on the ranking.

### 3) For Each Connection Approach, Generating New Virtual Loop Regions Successively, Exhausting All Combinations of Generation Orders, Creating the Corresponding Spatial Relationships, Determining the Connected Chemical Topological Structures, Calculating the Formation Probability of the Target Topological Structure in This Connection Approach, and Determining the Length Ranges of the Newly-Generated Loop Regions

After all possible splitting approaches and connection approaches are determined, it is possible to carry out the next step of design for each of the possible connection approaches based on the scores and ranks in step 2).

Again, the design of a protein [2] catenane structure is taken as an example below for illustration.

As shown in FIG. 3, each of the secondary structural motifs in the protein is numbered (for example, the six secondary structural motifs in the protein may be numbered sequentially as A, B, C, D, E, and F), and the corresponding loop region is denoted by the numberings of the two secondary structural motifs that it connects, for example, the loop region connecting the secondary structural motif A and the secondary structural motif B is the loop region AB, and the loop region connecting the N-terminus and the C-terminus in the original structure of the protein-of-interest is the loop region FA. A pair of loop regions (specifically loop region FA/loop region DE) is split. In order to form a catenane structure, there is only one possible connection approach, namely, regenerating a loop region DA and a loop region FE. Subsequently, virtual loop regions are generated respectively, and all combinations of generation orders are exhausted.

The virtual loop regions are generated by the algorithm for calculating the minimum solvent accessible path. The “solvent accessible path” is as defined above. Taking this minimum solvent accessible path as a virtual loop region can ensure that the first generated virtual loop region is immediately adjacent to the protein surface, while the subsequently generated loop regions are on the same side of the protein surface and the first generated loop region. In this way, the generation order of the loop regions determines the relative spatial position of the loop regions. By exhausting all combinations of the generation orders of the virtual loop regions, the relative spatial positions of all loop regions can be exhausted and we assume that the total number is n (i.e., all combinations of the generation orders). Subsequently, the topological structure of the protein obtained from each combination of the spatial relationships is determined by a computing program calculating its Gauss linking number or knot invariant. The number of the protein [2] catenanes that can be formed is counted as m, and m/n is taken as the formation probability of the catenane structures in the current connection approach. Meanwhile, the modification methods for proteins with the target chemical topological structure and the length ranges of the corresponding newly-generated virtual loop regions are also provided in this design process.

The programmatic design method provided in the present disclosure greatly reduces the number of possibilities required to be exhausted throughout the design by such means as optimizing the splitting approaches of the system and scoring and evaluating all possible connection approaches, and improves the efficiency of designing topological proteins.

### 4) Designing the Sequences of the Newly-Generated Loop Regions of the Topological Protein

Based on the length ranges of the virtual loop regions calculated in step 3), combinations of lengths of the loop regions that are more aligned with the scoring criteria in step 2) are selected as the specific number of amino acids in each of the actual loop regions, and the amino acid sequences of the actual loop regions are thereby designed.

Specifically, the amino acid sequences of the actual loop regions may be designed by any one of the following three design methods: (a) directly designing a flexible linking loop region with the target number of amino acids, wherein this loop region comprises any one of an enzyme cleavage site, an affinity purification tag, a residual motif after a coupling reaction, part or full sequence of the original loop region, or linking sequences consisting of glycine G and serine S, or any combination thereof: (b) searching structures similar to the two termini of the secondary structural motifs to be connected in PDB by any similar structural motif search algorithm of MASTER (Protein Sci. 2015, 24, 508-524), FragBag (PNAS 2010, 107, 3481-3486) or TOPOFIT (Protein Sci. 2004, 13, 1865-1874), and selecting the lengths of the loop regions that meet the requirement as the sequences of the target loop regions; or (c) designing the sequences of the linking loop regions with the target lengths by a computer-assisted means, such as any one of a Rosetta loop modelling method (Nat. Method 2009, 6, 551-552), a SCUBA method (Nature 2022, 602, 523-528) or a FoldX LoopReconstruction method (https://foldxsuite.crg.eu/command/LoopReconstruction).

### Definitions of the Invention

The terms used herein are chosen to best explain the principles and practical applications of the examples, or improvements over the technologies in the market, or to enable other persons of ordinary skill in the art to understand the examples disclosed herein. Unless otherwise defined, all technical and scientific terms used herein have the same meanings as conventionally understood by a person skilled in the art. For the sake of the present disclosure, the following terms are defined.

The term “about”, when used in combination with a numerical value, is intended to encompass numerical values in a range having a lower limit less than 5% of the specified numerical value and an upper limit greater than 5% of the specified numerical value.

The term “and/or”, when used to connect two or more options, shall be understood to mean either of or any two or more of the options.

As used herein, the term “comprising” is intended to include the elements, integers or steps, without the exclusion of any other elements, integers or steps. The term “comprising”, when used herein, also covers situations of consisting of the recited elements, integers or steps, unless otherwise indicated.

The numerical range represented by the term “numerical value A to numerical value B” refers to the range including the endpoint values A and B.

The numerical range represented by the term “or more” or “or less” refers to the range including this number itself.

The term “may” involves both the meaning of doing something and the meaning of not doing something.

The term “optional” or “optionally” means that factors such as some substances, components, execution steps, and imposed conditions may or may not be used.

The term “protein [2] catenane” indicates a protein catenane containing two mechanically interlocked rings, which is also referred to as a protein two-component link.

The terms “trefoil knot, 4₁ knot, 5₁ knot, 5₂ knot” indicate four different knot structures defined by the standard nomenclature in the knot theory. The trefoil knot indicates a knot with the minimum crossing number of 3 after projected on a two-dimensional plane: the 4₁ knot indicates a knot with the minimum crossing number of 4 after projected on a two-dimensional plane: the 5₁ knot indicates a symmetrical knot with the minimum crossing number of 5 after projected on a two-dimensional plane and the maximum symmetry of C5; and the 5₂ knot indicates a symmetrical knot with the minimum crossing number of 5 after projected on a two-dimensional plane and the maximum symmetry of C2.

Phrases such as “some specific/preferred embodiments”, “other specific/preferred embodiments”, and “embodiments” mentioned in the present specification mean that particular elements (for example, features, structures, properties and/or characteristics) described in relation to this embodiment are included in at least one of the embodiments described herein, and may or may not exist in other embodiments. Additionally, it should be appreciated that the elements may be combined in any suitable manner into various embodiments.

### Advantageous Effects of the Invention

As is clear from the technical solutions of the present disclosure, they have the following advantageous effects as compared to the prior art:

(1) The design method for topological proteins provided in the present disclosure provides a desirable platform for illustrating the effect of topological structures in the structure-activity relationships of proteins and also provides a convenient method for developing topologically functional proteins.

(2) The present disclosure provides a design strategy for topological isoforms widely applicable to a plurality of proteins. The whole strategy and design process are clear, easy to operate, and can be fully programmed. This strategy combined with an appropriate cyclization strategy can lead to the effective synthesis of topological proteins.

To render the above and other purposes, features, and advantages of the present disclosure more apparent and understandable, preferred examples are particularly described in detail below with reference to the drawings of the specification:

### BRIEF DESCRIPTION OF THE DRAWINGS

FIG. 1 shows a schematic diagram for a topological modification process of a protein.

FIG. 2 shows the statistical charts of the loop regions of all natural proteins, which is used for evaluating the rewiring approaches of protein motifs. (A) shows a statistical chart of the probability of generating the loop regions corresponding to the Euclidean distance between the secondary motifs to be connected; and (B) shows a statistical chart of the length distribution of the loop regions corresponding to the Euclidean distance between the secondary motifs to be connected.

FIG. 3 shows a schematic flow diagram for converting a protein-of-interest into a catenane, in which the short dotted lines indicate possible connection relationships and the long dotted lines indicate the generated virtual loop regions.

FIG. 4 shows diagrams for the crystallographic structure and topology diagram of a wild-type GFP.

FIG. 5 shows a schematic diagram for splitting a wild-type GFP and reconnecting the secondary structural motifs to form a catenane GFP, in which the virtual loop regions are indicated by dotted lines.

FIG. 6 shows the characterization of the physical and chemical properties of a catenane GFP and the proof of topology. (A) shows a schematic diagram for demonstrating the catenane structure by TEV protease cleavage; (B) shows the SDS-PAGE characterization of the catenane GFP and the products obtained after TEV protease digestion: and (C) shows mass spectrometry characterization of the molecular weight of a catenane GFP.

FIG. 7 shows the fluorescence spectra for a wild-type GFP and a catenane GFP.

FIG. 8 shows the crystallographic structure and topology diagram of a wild-type DHFR.

FIG. 9 shows a schematic diagram for splitting a wild-type DHFR and reconnecting the secondary motifs to form a catenane DHFR, in which the virtual loop regions are indicated by dotted lines.

FIG. 10 shows the characterization of the physical and chemical properties of a catenane DHFRe and the proof of topology. (A) shows the SDS-PAGE characterization of the catenane DHFR and the products obtained after TEV protease digestion; and (B) shows mass spectrometry characterization of the molecular weight of a catenane DHFR.

FIG. 11 shows a reaction kinetic profile of catalyzing reduction of the substrate dihydrofolic acid to tetrahydrofolic acid by a wild-type DHFR and a catenane DHFR.

FIG. 12 shows the crystallographic structure and topology diagram of a wild-type Spy.

FIG. 13 shows a schematic diagram for splitting a wild-type Spy and reconnecting the secondary motifs to form a catenane Spy, in which the virtual loop regions are indicated by dotted lines.

FIG. 14 shows a schematic diagram for splitting a wild-type Spy and reconnecting the secondary motifs to form a pseudorotaxane Spy, in which the virtual loop regions are indicated by dotted lines.

FIG. 15 shows the characterization of the physical and chemical properties of a catenane Spy and a pseudorotaxane Spy and the proof of topology. (A) shows mass spectrometry characterization of the catenane Spy: (B) shows mass spectrometry characterization of the pseudorotaxane Spy: and (C) shows the SDS-PAGE characterization of the catenane Spy and the product obtained after TEV protease digestion.

FIG. 16 shows a schematic diagram for splitting a wild-type Spy and reconnecting the secondary motifs to form a lasso Spy, in which the virtual loop regions are indicated by dotted lines.

FIG. 17 shows a schematic diagram for splitting a wild-type Spy and reconnecting the secondary motifs to form a knot Spy, in which the virtual loop regions are indicated by dotted lines.

FIG. 18 shows characterization of the physical and chemical properties of a lasso Spy and a knot Spy and the proof of topology. (A) shows mass spectrometry characterization of the lasso Spy: and (B) shows mass spectrometry characterization of the knot Spy.

FIG. 19 shows a schematic diagram for splitting a wild-type Spy and reconnecting the secondary motifs to form a cyclic Spy, in which the virtual loop regions are indicated by dotted lines.

FIG. 20 shows a schematic diagram for splitting a wild-type Spy and reconnecting the secondary motifs to form a linear Spy, in which the virtual loop regions are indicated by dotted lines.

FIG. 21 shows characterization of the physical and chemical properties of a cyclic Spy and a linear Spy and the proof of topology. (A) shows mass spectrometry characterization of the cyclic Spy: and (B) shows mass spectrometry characterization of the linear Spy.

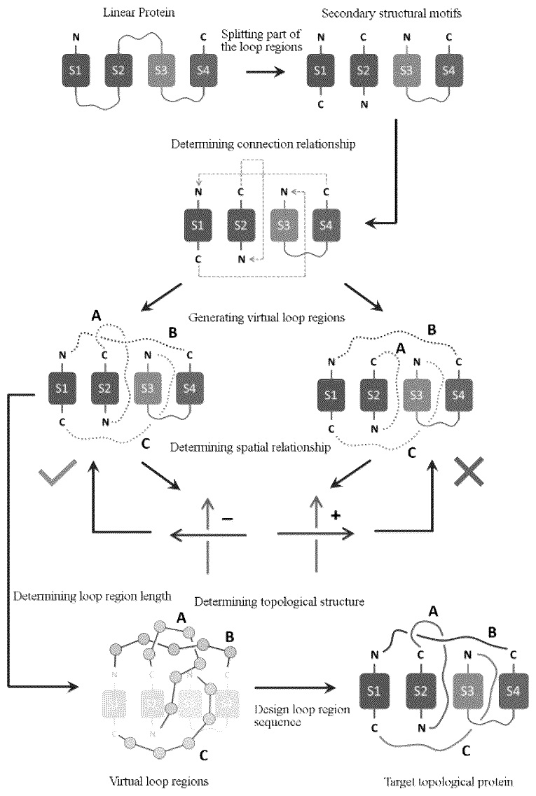

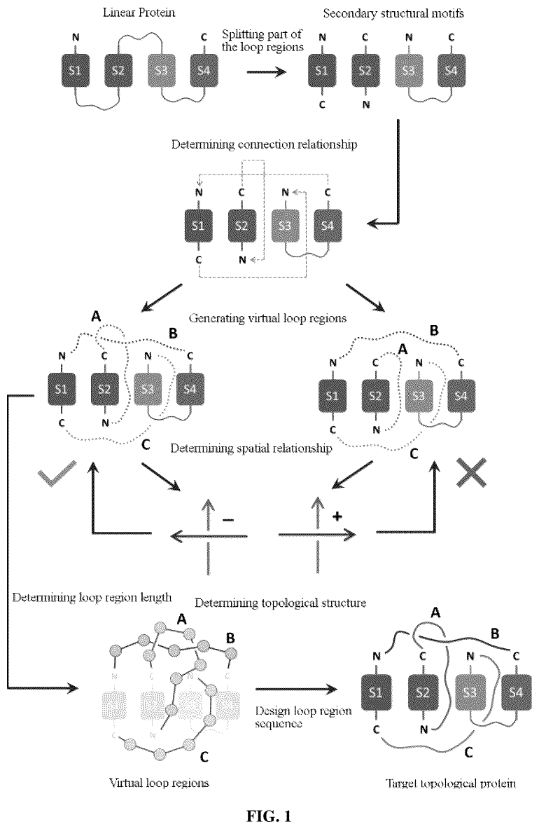

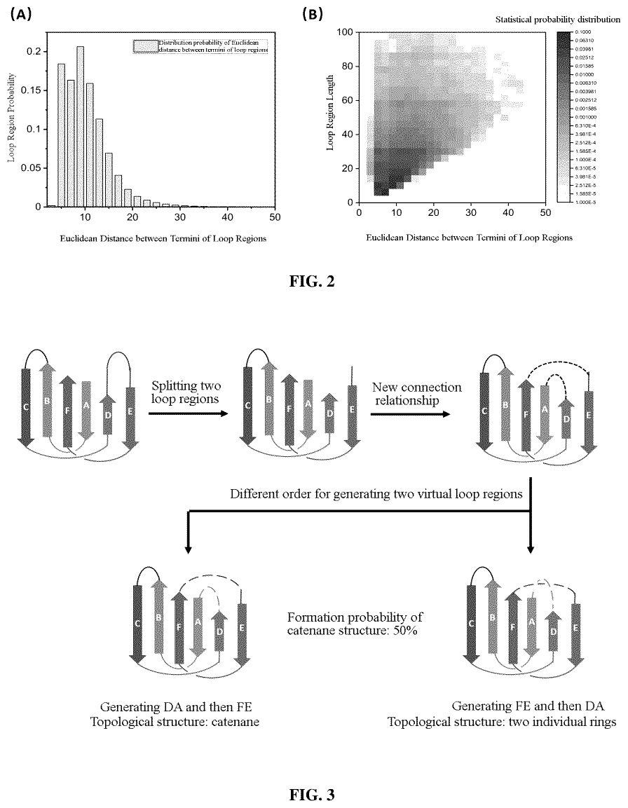

### DETAILED DESCRIPTION

The specific examples listed herein are only exemplary examples of the present disclosure, and the present disclosure is not limited to the specific examples described below. For a person skilled in the art, any equivalent modifications and substitutions to the examples described below are also within the scope of the present disclosure. Therefore, all equivalent alterations and modifications made without departing from the spirit and scope of the present disclosure shall be encompassed in the scope of the present disclosure.

The experimental materials, experimental reagents, and instruments used in the examples of the present disclosure are all commercially available.

### Example 1: Design of Single-Domain Catenane Green Fluorescent Protein (GFP)

The crystallographic structure (PDB No.: 6DQ1) and the topology diagram of a wild-type GFP were as shown in FIG. 4.

Following the design idea of the design method for topological proteins provided in the present disclosure, there were the following possible designs for making the catenane GFP (taking split of two loop regions as an example here):

Firstly, the wild-type GFP was analyzed and found to contain eleven secondary structural motifs (named secondary structural motif 1, secondary structural motif 2 . . . secondary structural motif 10, and secondary structural motif 11, respectively) and eleven loop regions (including the virtual loop region connecting the N-terminus and the C-terminus). According to the approach of splitting two loop regions of the protein [2] catenane provided in step i) of the programmatic design method for the topological protein provided in the present disclosure, the two loop regions in the wild-type GFP were rationally split. There were a total of 55 splitting approaches (the specific calculation method: 11×(11−1)/2=55), and each splitting approach corresponded to only one new connection approach, so there were eventually 55 different connection approaches, accordingly. Thereafter, each of the connection approaches was evaluated based on the evaluation method in step ii), and the structures with high scores were selected for design.

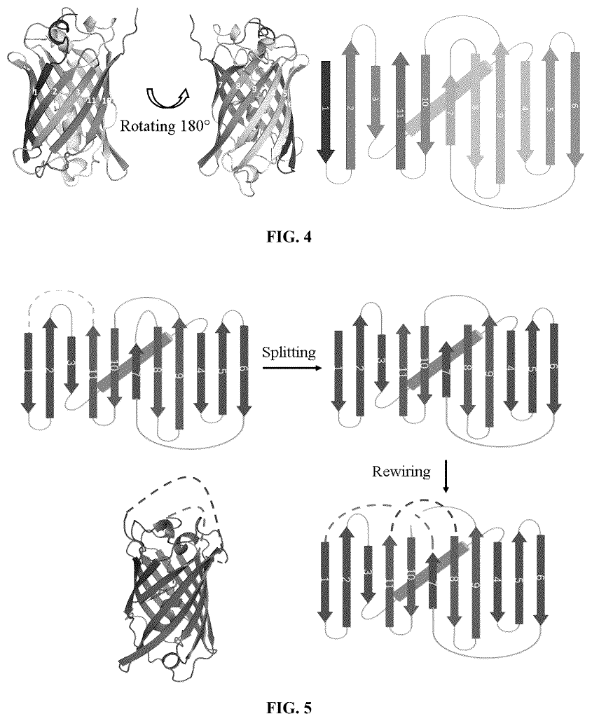

As shown in FIG. 5, the virtual loop region between the N-terminus and the C-terminus and the loop region connecting the secondary structural motif 7 and the secondary structural motif 8 of GFP were removed, and rewiring was performed between the secondary structural motif 7 and the secondary structural motif 1 and between the secondary structural motif 11 and the secondary structural motif 8.

Subsequently, according to the operations of step iii) in the programmatic design method for the topological protein provided in the present disclosure, a virtual loop region was generated at the above two positions to be connected, and all combinations of the generation orders were exhausted. The topological structures were determined for the resulting structures and the catenane formation probability was calculated. The optimal design approach of the catenane GFP was finally obtained (with the formation probability of 50%), as shown in FIG. 5.

Furthermore, the following operations were carried out according to step iv) of the programmatic design method for the topological protein provided in the present disclosure: based on the length ranges of the newly-generated actual loop regions, a combination of lengths of the loop regions that were more aligned with the basis for scoring and evaluation disclosed in the specification of the present disclosure was selected as the specific number of the amino acids in each of the actual loop regions, and the amino acid sequences of the actual loop regions were thereby designed, whereby the sequence included a combination of an enzyme cleavage site, an affinity purification tag, a residual motif after a coupling reaction, and linking sequences consisting of glycine G and serine S. The two rings constituting this catenane structure were composed of β-sheets 1 to 7 and β-sheets 8 to 11 of GFP, respectively. The topological structure and the corresponding secondary structure numberings of the catenane GFP were as shown in FIG. 5.

The amino acid sequence of the wild-type GFP was as shown below:

(SEQ ID NO: 1)
MGSSMSKGEELFTGVVPILVELDGDVNGHKFSVRGEGEGDATIGKL
TLKFICTTGKLPVPWPTLVTTLTYGVQCFSRYPDHMKRHDFFKSAM
PEGYVQERTISFKDDGKYKTRAVVKFEGDTLVNRIELKGTDFKEDG
NILGHKLEYNFNSHNVYITADKQKNGIKANFTVRHNVEDGSVQLAD
HYQQNTPIGDGPVLLPDNHYLSTQTVLSKDPNEKRDHMVLLEFVTA
AGITHGMDELYKLEHHHHHH.

The sequences shown in bold were the introduced sequence (LE) translated by the DNA endonuclease involved in molecular cloning, the starting amino acid residue and the flexible loop region consisting of G and S (MGSS), and the affinity purification tag (HHHHHH), respectively.

The sequence information of ring 1 and ring 2 in catenane GFP resulting from topological reconstruction was as shown below. The underlined parts were the sequences as same as those of the wild-type GFP (specifically, the amino acid sequences at positions 1 to 156 and at positions 157 to 238 of the wild-type GFP, respectively), and the bold parts were the introduced sequences of the newly-generated loop regions as compared to the wild-type GFP, which primarily included a sequence (GT/TS/EL) translated by the DNA endonuclease involved in molecular cloning, a Tobacco Etch Virus (TEV) protease cleavage site (ENLYFQG) for proving the topological structure, a recognition polypeptide sequence for cyclization (LPETG), and a flexible loop region consisting of G and S.

Ring 1:

(SEQ ID NO: 2)
MSKGEELFTGVVPILVELDGDVNGHKFSVRGEGEGDATIGKLTLK
FICTTGKLPVPWPTLVTTLTYGVQCFSRYPDHMKRHDFFKSAMPE
GYVQERTISFKDDGKYKTRAVVKFEGDTLVNRIELKGTDFKEDGN
ILGHKLEYNFNSHNVYITADKGTCFNGGENLYFQGAS

Ring 2:

(SEQ ID NO: 3)
QKNGIKANFTVRHNVEDGSVQLADHYQQNTPIGDGPVLLPDNHYL
STQTVLSKDPNEKRDHMVLLEFVTAAGITHGMDELYKTSLPETGG
GEL

After synthesis, the basic physical and chemical properties of the catenane GFP were characterized.

First of all, the catenane GFP was subjected to the TEV protease cleavage experiment to verify its topological structure. The experimental method and specific operations were as follows:

To a stock solution of catenane GFP at a concentration of 20 μM, 20× enzyme cleavage buffer (1 M Tris-HCl, 2 M NaCl, 20 mM EDTA, 100 mM DTT, pH=8.0) and one-tenth equivalent of TEV protease were added, and the final volume was adjusted with sterile water. The mixture was incubated at 30° C. for 2 h. Afterwards, 5× loading buffer (250 mM Tris, pH 6.8, 10% SDS, 30% glycerol, 5% β-mercaptoethanol, 0.02% bromophenol blue) was added and mixed evenly, and then boiled at 98° C. for 10 min. Vertical electrophoresis was run using 15% sodium dodecyl sulfate-polyacrylamide gel in a mini vertical electrophoresis cell with the Tris-glycine system (6.06 g of Tris, 36.03 g of glycine, 2 g of SDS, ddH₂O added to adjust the volume to 2 L) as the buffer. Electrophoresis was run first in the stacking gel zone at a voltage of 90 V. After the sample was concentrated into a thin line and completely entered the resolving gel zone, the voltage was adjusted to 140 V until the bromophenol blue indicator dye at the leading edge was close to the boundary of the resolving gel. After completion of the electrophoresis, the glass plate outside the gel was removed, and the gel was dyed with Coomassie Blue Staining Solution (50% ddH₂O, 40% methanol, 10% glacial acetic acid, 0.1% Coomassie Blue R250). After dyeing, the floating color was removed with a destaining solution (50% ddH₂O, 40% methanol, 10% glacial acetic acid). The gel was imaged with a gel imaging system.

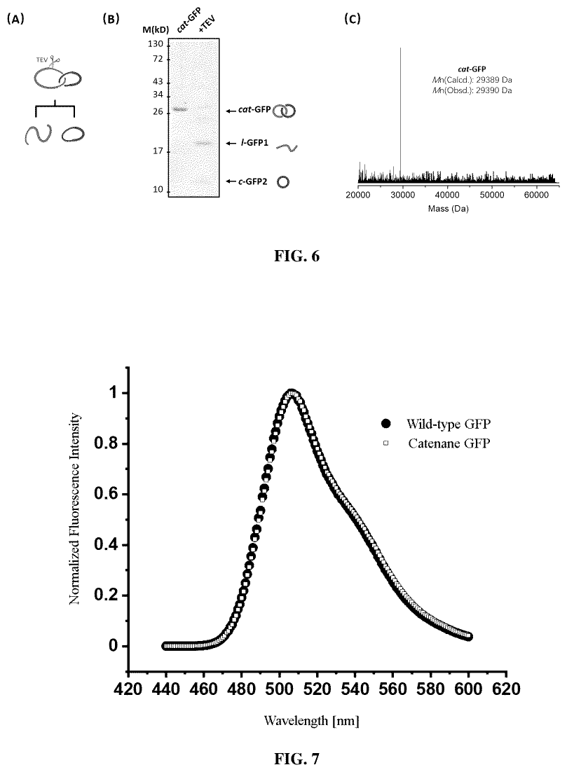

Experimental results: the results showed that the synthesized proteins met the requirement for the molecular weight of the target catenane structure, and the results of the TEV protease cleavage also showed that the linear l-GFP1 and cyclic c-GFP2 (as shown in (B) of FIG. 6) were obtained, demonstrating that the protein-of-interest had the bicyclic interlocked structure of the catenane.

The molecular weight of catenane GFP was subsequently confirmed by high-performance liquid chromatography-electrospray mass spectrometry (LC-MS) (as shown in (C) of FIG. 6), and was as expected. The mass spectrometry conditions and operation methods in this experiment were as follows: the sample concentration was 0.5 mg/mL, the Acquity UPLC H-Class system and the Acquity QDa mass spectrometry detector were used for characterization, and the MassLynx V4.1 analysis software was used for integration.

Thereafter, the fluorescent properties of the catenane GFP were characterized by the fluorescence spectrometry. The experimental conditions and operation methods were as follows: the catenane GFP and the wild-type GFP at a concentration of 5 μM were dissolved in PBS buffer separately. The comparison between the fluorescence spectra of the catenane GFP and the wild-type GFP was as shown in FIG. 7: the maximum emission wavelengths of both the wild-type GFP and the catenane GFP, when excited at a wavelength of 395 nm, were around 510 nm, indicating that the fluorescent properties of the single-domain catenane GFP were maintained.

### Example 2: Design of Single-Domain Catenane Dihydrofolate Reductase (DHFR)

The crystallographic structure (PDB No.: 4KJJ) and the topology diagram of a wild-type DHFR were as shown in FIG. 8.

Following the design idea of the design method for topological proteins provided in the present disclosure, the catenane DHFR was designed as follows:

The wild-type DHFR was analyzed and found to contain twelve secondary structural motifs (named secondary structural motif 1, secondary structural motif 2 . . . secondary structural motif 11, and secondary structural motif 12, respectively) and twelve loop regions (including the virtual loop region connecting the N-terminus and the C-terminus). According to the approach of splitting two loop regions of the protein [2] catenane provided in step i) of the programmatic design method for the topological protein provided in the present disclosure, the two loop regions in the wild-type DHFR were rationally split. There were a total of 66 splitting approaches (the specific calculation method: 12×(12−1)/2=66), and each splitting approach corresponded to only one new connection approach, so there were eventually 66 different connection approaches, accordingly. Thereafter, each of the connection approaches was evaluated based on the evaluation method in step ii), and the structures with high scores were selected for design.

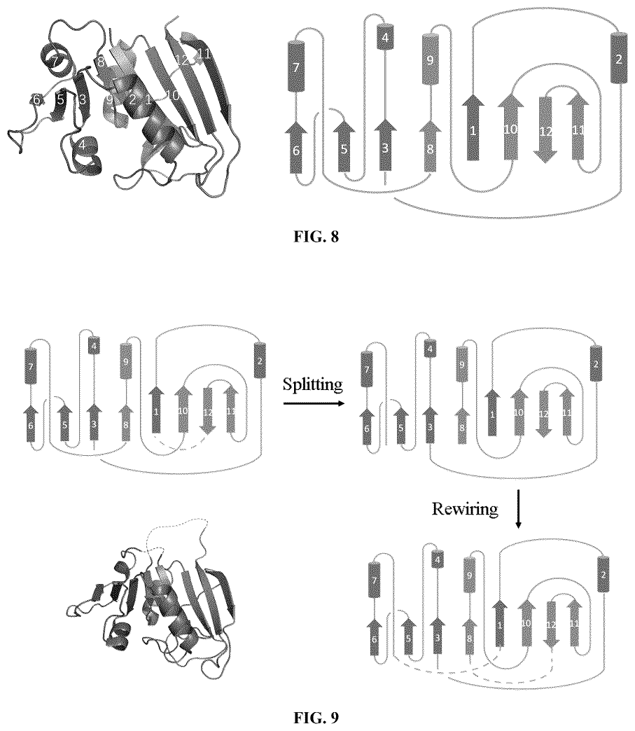

FIG. 9 showed the rewiring approach with the highest score among reconnections after splitting two loop regions of the wild-type DHFR. Specifically, FIG. 9 showed the removal of the loop region connecting the secondary structural motif 7 and the secondary structural motif 8 of DHFR and the virtual loop region at the N-terminus and C-terminus, and the rewiring between the secondary structural motif 7 and the secondary structural motif 1 and between the secondary structural motif 12 and the secondary structural motif 8.

Subsequently, according to the operations in step iii) of the programmatic design method for the topological protein provided in the present disclosure, a virtual loop region was generated at the above two positions to be connected, and all combinations of the generation orders were exhausted. The topological structures were determined for the resulting structures and the formation probability of the target topological structure was calculated. The optimal design approach of the catenane DHFR was finally screened out (with the formation probability of 100%), as shown in FIG. 9.

Furthermore, the following operations were carried out according to step iv) of the programmatic design method for the topological protein provided in the present disclosure: based on the length ranges of the newly-generated actual loop regions, the specific number of the amino acids in the actual loop region was preferably selected, and the actual loop region sequence was designed, whereby the sequence included a combination of an enzyme cleavage site, an affinity purification tag, a residual motif after a coupling reaction, and linking sequences consisting of glycine G and serine S. The two rings constituting this catenane structure were composed of the secondary structural motifs 1 to 7 and the secondary structural motifs 8 to 12 of DHFR, respectively. The topological structure and the secondary structural motif numberings of the catenane structure were as schematically shown in FIG. 9.

The amino acid sequence of the wild-type DHFR was as shown below:

(SEQ ID NO: 4)
MISLIAALAVDRVIGMENAMPWNLPADLAWFKRNTLNKPVIMGRH
TWESIGRPLPGRKNIILSSQPGTDDRVTWVKSVDEAIAACGDVPE
IMVIGGGRVYEQFLPKAQKLYLTHIDAEVEGDTHFPDYEPDDWES
VFSEFHDADAQNSHSYCFEILERRLEHHHHHH.

The sequences shown in bold were the introduced sequence (LE) translated by the DNA endonuclease involved in molecular cloning and the affinity purification tag (HHHHHH), respectively.

The sequence information of ring 1 and ring 2 in the catenane DHFR resulting from topological modification was as shown below. The underlined parts were the sequences as same as those of the wild-type DHFR (specifically, the amino acid sequences at positions 1 to 88 and at positions 89 to 159 of the wild-type DHFR, respectively), and the bold parts were the introduced sequences of the newly-generated loop regions as compared to the wild-type DHFR, which primarily included a sequence (AS/GT/TS/EL) translated by the DNA endonuclease involved in molecular cloning, a TEV protease cleavage site (ENLYFQG) for proving the topological structure, an affinity purification tag (HHHHHH), and a flexible loop region consisting of G and S.

Ring 1:

(SEQ ID NO: 5)
CFNGGENLYFQGASLPADLAWFKRNTLNKPVIMGRHTWESIGRPL
PGRKNIILSSQPGTDDRVTWVKSVDEAIAACGDVGGMISLIAALA
VDRVIGMENAMPWNGT

Ring 2:

(SEQ ID NO: 6)
CFNGGHHHHHHELPEIMVIGGGRVYEQFLPKAQKLYLTHIDAEVE
GDTHFPDYEPDDWESVESEFHDADAQNSHSYCFEILERRGGSGGT
S

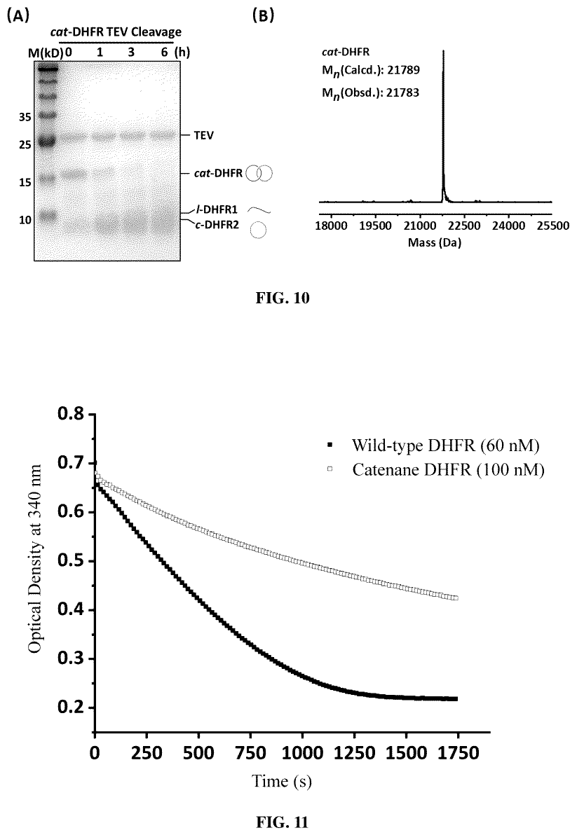

The catenane DHFR was expressed and purified and its basic physical and chemical properties were characterized. First of all, the catenane DHFR was subjected to the TEV protease cleavage experiment (the specific results were shown in (A) of FIG. 10). The experimental method and specific operations were as follows:

To a stock solution of catenane DHFR at a concentration of 20 μM, 20× enzyme cleavage buffer (1 M Tris-HCl, 2 M NaCl, 20 mM EDTA, 100 mM DTT, pH=8.0) and an equivalent of TEV protease were added, and the final volume was adjusted with sterile water. The mixture was incubated at 30° C. During incubation, the reaction of the mixed solution was quenched at a specific time point. 5× Loading buffer (250 mM Tris, pH 6.8, 10% SDS, 30% glycerol, 5% β-mercaptoethanol, 0.02% bromophenol blue) was added and mixed evenly, and then boiled at 98° C. for 10 min. Vertical electrophoresis was run using 15% sodium dodecyl sulfate-polyacrylamide gel in a mini vertical electrophoresis cell with the Tris-glycine system (6.06 g of Tris, 36.03 g of glycine, 2 g of SDS, ddH2O added to adjust the volume to 2 L) as the buffer. Electrophoresis was run first in the stacking gel zone at a voltage of 90 V. After the sample was concentrated into a thin line and completely entered the resolving gel zone, the voltage was adjusted to 140 V until the bromophenol blue indicator dye at the leading edge was close to the boundary of the resolving gel. After completion of the electrophoresis, the glass plate outside the gel was removed, and the gel was dyed with Coomassie Blue Staining Solution (50% ddH₂O, 40% methanol, 10% glacial acetic acid, 0.1% Coomassie Blue R250). After dyeing, the floating color was removed with a destaining solution (50% ddH₂O, 40% methanol, 10% glacial acetic acid). The gel was imaged with a gel imaging system.

In the case of catenane DHFR, there were some c-DHFR2 bands in samples without adding the TEV protease because the purified catenane protein inherently contained a portion of cyclic monomer c-DHFR2 (with Histag), which was slightly different from the main product in terms of the molecular weight and may have a non-specific interaction with the main product. Both the cyclic product c-DHFR2 and the linear product l-DHFR1 resulting from enzyme cleavage of the catenane DHFR had very small and very similar apparent molecular weights, and thus appeared as diffuse bands on the gel image (as shown in (A) of FIG. 10).

In FIG. 10, (B) showed the result of mass spectrometry characterization of the catenane DHFR, in which the protein samples were prepared into aqueous solutions having a concentration of about 0.5 mg/mL. The prepared protein samples were characterized by liquid chromatography-mass spectrometry using the Acquity UPLC H-Class system and the Acquity QDa mass spectrometry detector, and integrated using the MassLynx V4.1 analysis software to obtain the corresponding molecular weight. The resulting molecular weight was as expected, proving successful synthesis of the catenane DHFR.

Subsequently, the catalytic activity of the catenane DHFR was also characterized. Dihydrofolic acid could be catalytically reduced to tetrahydrofolic acid by DHFR in the presence of the cofactor nicotinamide adenine dinucleotide phosphate II (NADPH).

The experimental method and specific operations were as follows: a phosphate buffer (40.1 mM K₂HPO₄, 9.9 mM NaH₂PO₄, 5 mM β-mercaptoethanol, pH=7.5) was prepared. The coenzyme NADPH and the substrate dihydrofolic acid (DHF) were dissolved in the buffer to prepare 20 mM NADPH concentrate stock solution and 5 mM DHF concentrate stock solution. The coenzyme NADPH was diluted by the buffer into 0.5 mM, and the substrate DHF was diluted into 0.33 mM. The DHFR samples to be tested were diluted with the buffer. As the enzymatic activity of the wild-type DHFR was higher than that of the catenane DHFR, for ease of reaction detection and comparison, the wild-type DHFR was diluted into 60 nM, and the catenane DHFR was diluted into 100 nM. 100 μL of DHFR diluted samples were mixed with 40 μL of NADPH (0.5 mM) and 60 μL of DHF (0.33 mM) (both of which were in excess) in a transparent 96-well plate, and then the mixture was immediately placed on a microplate reader for real-time monitoring of the change in optical density of the solution at 340 nm to obtain the kinetic profile of the enzyme-catalyzed reaction.

Experimental results: the kinetic profiles of the catalytic reduction of the substrate dihydrofolic acid by the catenane DHFR and the wild-type DHFR were as shown in FIG. 11. Decrease in the optical density at 340 nm reflected the consumption of the nicotinamide adenine dinucleotide phosphate II and indicated the reaction course, suggesting that the single-domain DHFR was still catalytically active.

### Example 3: Design of Single-Domain Catenane Spy and Pseudorotaxane Spy

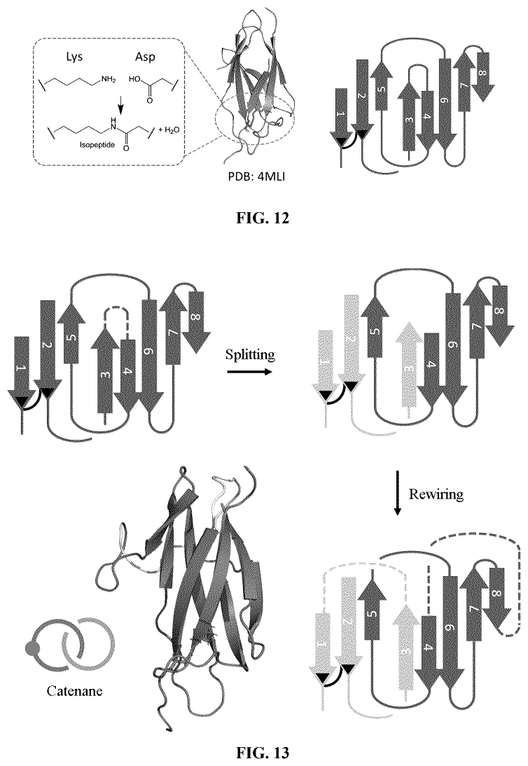

The wild-type Spy was derived from the CnaB2 domain. Its crystallographic structure (PDB No.: 4MLI) and topology diagram were as shown in FIG. 12. Its crystallographic structure contained SpyTag and SpyCatcher. The Asp on the SpyTag could undergo an autocatalytic reaction with the Lys on the SpyCatcher to form a natural isopeptide bond that joined the two parts together. The isopeptide bond was regarded as an effectively connected loop region in design of the present example.

Following the design idea of the design method for topological proteins provided in the present disclosure, the catenane Spy was designed as follows:

The wild-type Spy structure was analyzed and found to contain eight secondary structural motifs (named secondary structural motif 1, secondary structural motif 2, . . . , secondary structural motif 7, and secondary structural motif 8, respectively) and eight loop regions (including the virtual loop regions connecting the N-terminus and the C-terminus). According to the approach of splitting two loop regions of the protein [2] catenane provided in step i) of the programmatic design method for the topological protein provided in the present disclosure, the loop regions in the wild-type Spy were rationally split. There were a total of 28 splitting approaches (the specific calculation method: 8×(8−1)/2=28), and each splitting approach corresponded to only one new connection approach, so there were eventually 28 different connection approaches, accordingly. Thereafter, each of the connection approaches was evaluated based on the evaluation method in step ii), and the structures with high scores were selected for design.

FIG. 13 showed the rewiring approach with the highest score among reconnections after splitting two loop regions of the wild-type Spy. Specifically, FIG. 13 showed the removal of the loop region connecting the secondary structural motif 3 and the secondary structural motif 4 of Spy, and the rewiring between the secondary structural motif 1 and the secondary structural motif 3 and between the secondary structural motif 4 and the secondary structural motif 8.

Subsequently, according to the operations of step iii) in the programmatic design method for the topological protein provided in the present disclosure, a virtual loop region was generated at the above two positions to be connected, and all combinations of the generation orders were exhausted. The topological structures were determined for the resulting structures and the formation probability of the target topological structure was calculated. The optimal design approach of the catenane Spy was finally screened out (with the formation probability of 80%), as shown in FIG. 13.

Furthermore, the following operations were carried out according to step iv) of the programmatic design method for the topological protein provided in the present disclosure: based on the length ranges of the newly-generated actual loop regions, a combination of lengths of the loop regions that were more aligned with the basis for scoring and evaluation disclosed in the specification of the present disclosure was selected as the specific number of the amino acids in each of the actual loop regions, and the amino acid sequences of the actual loop regions were thereby designed, whereby the sequence included a combination of an enzyme cleavage site, an affinity purification tag, a residual motif after a coupling reaction, and linking sequences consisting of glycine G and serine S. The two rings constituting this catenane structure were composed of β-sheets 1 to 3 and β-sheets 4 to 7 of Spy, respectively. The topological structure and the corresponding secondary structural motif numberings of the catenane structure were as shown in FIG. 13.

The amino acid sequence of the wild-type Spy was as shown below:

(SEQ ID NO: 7)
MGSSAHIVMVDAYKPTKGEDSATHIKFSKRDEDGKELAGATMELR
DSGKTISTWISDGQVKDFYLYPGKYTFVETAAPDGYEVATAITFT
VNEQGQVTVNGKATKLEHHHHHH.

The sequences shown in bold were the introduced leader sequence (MGSS) expressed in molecular cloning, the sequence (LE) translated by the DNA endonuclease, and the affinity purification tag (HHHHHH), respectively.

The sequence information of ring 1 and ring 2 in the catenane Spy resulting from topological modification was as shown below. The underlined parts were the sequences as same as those of the wild-type Spy (specifically, the amino acid sequences at positions 14 to 42,at positions 1 to 13, and at positions 43 to 101 of the wild-type Spy, respectively), and the bold parts were the introduced sequences of the newly-generated loop regions as compared to the wild-type Spy, which primarily included a sequence (GT/EL/AS/RS) translated by the DNA endonuclease involved in molecular cloning, a TEV protease cleavage site (ENLYFQG) for proving the topological structure, an affinity purification tag (HHHHHH), and a flexible loop region consisting of G and S.

Ring 1:

(SEQ ID NO: 8)
GGGELGEDSATHIKESKRDEDGKELAGATMELRDRSGGSGGSENL
YFQGGSGGSAHIVMVDAYKPTKGGSGGSG

Ring 2:

(SEQ ID NO: 9)
CFNASHHHHHHSGKTISTWISDGQVKDFYLYPGKYTFVETAAPDG
YEVATAITFTVNEQGQVTVNGKATKGGSGGSGGSGT

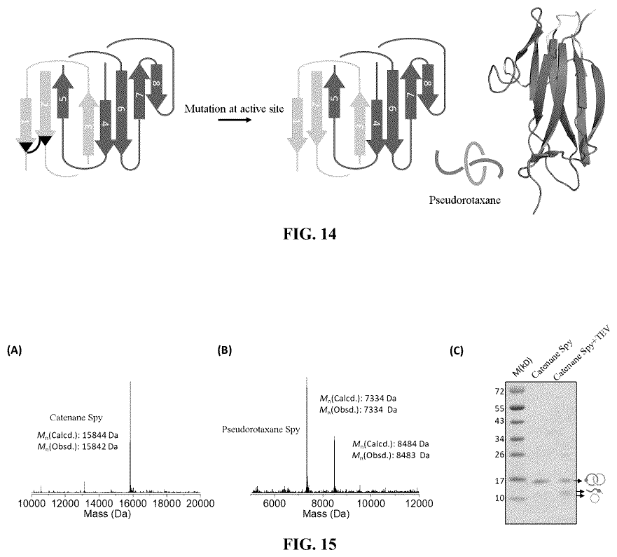

As shown in FIG. 14, the original isopeptide bond connectivity could be removed by mutating the active site Asp (aspartic acid, D) for forming the isopeptide bond in the catenane Spy to Ala (alanine, A) to obtain a pseudorotaxane Spy, in which the linear secondary structural motifs 1 to 3 constituted the axis of the pseudorotaxane Spy and the secondary structural motifs 4 to 8 constituted the ring component of the pseudorotaxane Spy.

The sequence information of the axis and ring components of the pseudorotaxane Spy was as shown below. The underlined parts were the sequences as same as those of the wild-type Spy (specifically, the amino acid sequences at positions 14 to 42, at positions 1 to 6 and 8 to 13,and at positions 43 to 101 of the wild-type Spy, respectively); the shadow was the mutated active site at position 7; and the bold parts were the introduced sequences of the newly-generated loop regions as compared to the wild-type Spy, which primarily included a sequence (GT/EL/AS/RS) translated by the DNA endonuclease involved in molecular cloning, a TEV protease cleavage site (ENLYFQG) for proving the topological structure, an affinity purification tag (HHHHHH), and a flexible loop region consisting of G and S.

Pseudorotaxane axis:

(SEQ ID NO: 10)
GGGELGEDSATHIKFSKRDEDGKELAGATMELRDRSGGSGGSENLYFQGGSGG

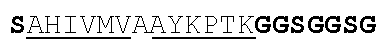

Pseudorotaxane ring:

(SEQ ID NO: 9)
CFNASHHHHHHSGKTISTWISDGQVKDFYLYPGKYTFVETAAPDG
YEVATAITFTVNEQGQVTVNGKATKGGSGGSGGSGT

The catenane Spy and pseudorotaxane Spy were expressed and purified, and their basic physical and chemical properties were characterized.

Firstly, the molecular weights of the two products were confirmed by high-performance liquid chromatography-electrospray mass spectrometry (LC-MS). The mass spectrometry conditions and operation methods in this experiment were as follows: the sample concentration was 0.5 mg/mL, the Acquity UPLC H-Class system and the Acquity QDa mass spectrometry detector were used for characterization, and the MassLynx V4.1 analysis software was used for integration.

Thereafter, the catenane Spy was subjected to the TEV protease cleavage experiment (the specific result was shown in (C) of FIG. 15). The experimental method and specific operations were as follows:

To a stock solution of the catenane Spy at a concentration of 20 μM, 20× enzyme cleavage buffer (1 M Tris-HCl, 2 M NaCl, 20 mM EDTA, 100 mM DTT, pH=8.0) and an equivalent of TEV protease were added, and the final volume was adjusted with sterile water. The mixture was incubated at 30° C. During the incubation, the reaction of the mixed solution was quenched at a specific point in time. 5× Loading buffer (250 mM Tris, pH 6.8, 10% SDS, 30% glycerol, 5% β-mercaptoethanol, 0.02% bromophenol blue) was added and mixed evenly, and then boiled at 98° C. for 10 min. Vertical electrophoresis was run using 15% sodium dodecyl sulfate-polyacrylamide gel in a mini vertical electrophoresis cell with the Tris-glycine system (6.06 g of Tris, 36.03 g of glycine, 2 g of SDS, ddH₂O added to adjust the volume to 2 L) as the buffer. Electrophoresis was run first in the stacking gel zone at a voltage of 90 V. After the sample was concentrated into a thin line and completely entered the resolving gel zone, the voltage was adjusted to 140 V until the bromophenol blue indicator dye at the leading edge was close to the boundary of the resolving gel. After completion of the electrophoresis, the glass plate outside the gel was removed, and the gel was dyed with Coomassie Blue Staining Solution (50% ddH₂O, 40% methanol, 10% glacial acetic acid, 0.1% Coomassie Blue R250). After dyeing, the floating color was removed with a destaining solution (50% ddH₂O, 40% methanol, 10% glacial acetic acid). The gel was imaged with a gel imaging system.

Experimental results: the results showed that the synthesized proteins met the requirements for the molecular weights of the target catenane structure and the pseudorotaxane structure (as shown in (A) and (B) of FIG. 15), and the results of the TEV protease cleavage also showed the linear product and cyclic product as anticipated (as shown in (C) of FIG. 15, but only part of enzyme cleavage was achieved because the TEV recognition site was in a conformationally restricted, non-extendable state after completion of the reaction), demonstrating that the protein-of-interest had the bicyclic interlocked structure of the catenane.

### Example 4: Design of Single-Domain Lasso Spy and Knot Spy

The wild-type Spy was derived from the CnaB2 domain. Its crystallographic structure (PDB No.: 4MLI) and topology diagram were as shown in FIG. 12. Its crystallographic structure contained SpyTag and SpyCatcher.

The Asp on SpyTag could undergo an autocatalytic coupling reaction with the Lys on SpyCatcher to form a natural isopeptide bond that joined the two parts together. The isopeptide bond was regarded as an effectively connected loop region in the design of the present example.

Following the design idea of the design method for topological proteins provided in the present disclosure, the lasso Spy was designed as follows:

The wild-type Spy structure was analyzed and found to contain eight secondary structural motifs (named secondary structural motif 1, secondary structural motif 2, . . . , secondary structural motif 7, and secondary structural motif 8, respectively) and eight loop regions (including the virtual loop region connecting the N-terminus and the C-terminus). According to the approach of splitting two loop regions of the lasso protein provided in step i) of the programmatic design method for the topological protein provided in the present disclosure, the loop regions in the wild-type Spy were rationally split. There were a total of 28 splitting approaches (the specific calculation method: 8×(8−1)/2=28), and each splitting approach corresponded to only one new connection approach, so there were eventually 28 different connection approaches, accordingly. Thereafter, each of the connection approaches was evaluated based on the evaluation method in step ii), and the structures with high scores were selected for design.

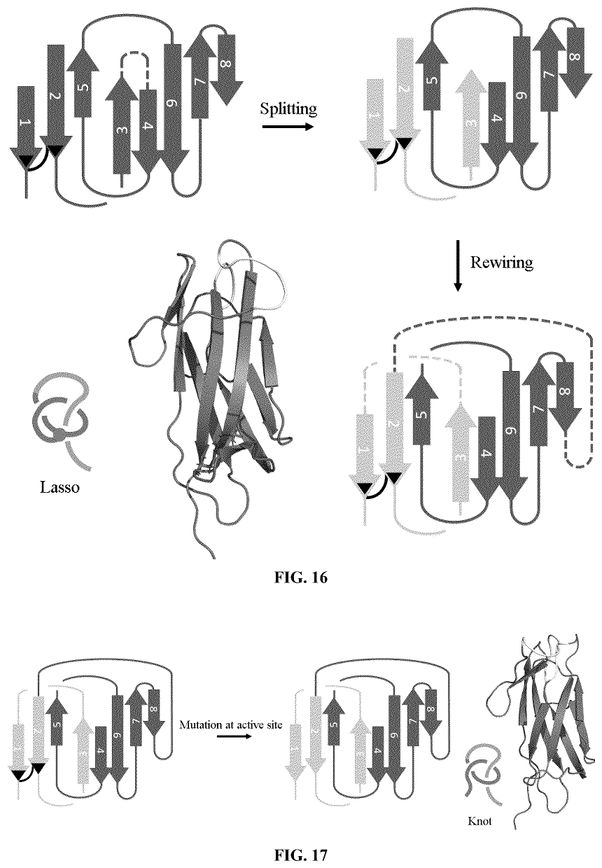

FIG. 16 showed the rewiring approach with the highest score among reconnections after splitting two loop regions of the wild-type Spy. Specifically, FIG. 16 showed the removal of the loop region connecting the secondary structural motif 3 and the secondary structural motif 4 of Spy, and the rewiring between the secondary structural motif 1 and the secondary structural motif 3 and between the secondary structural motif 2 and the secondary structural motif 8.

Subsequently, according to the operations of step iii) in the programmatic design method for the topological protein provided in the present disclosure, a virtual loop region was generated at the above two positions to be connected, and all combinations of the generation orders were exhausted. The topological structures were determined for the resulting structures and the formation probability of the target topological structure was calculated. The optimal design approach of the lasso Spy was finally screened out (with the formation probability of 50%), as shown in FIG. 16.

Furthermore, the following operations were carried out according to step iv) of the programmatic design method for the topological protein provided in the present disclosure: based on the length ranges of the newly-generated actual loop regions, a combination of lengths of the loop regions that were more aligned with the basis for scoring and evaluating disclosed in the specification of the present disclosure was selected as the specific number of the amino acids in each of the actual loop regions, and the amino acid sequences of the actual loop regions were thereby designed, whereby the sequence included a combination of an enzyme cleavage site, an affinity purification tag, a residual motif after a coupling reaction, and linking sequences consisting of glycine G and serine S. The topological structure and the corresponding secondary structural motif numberings of the lasso Spy structure were as shown in FIG. 16.

The amino acid sequence of the wild-type Spy was as shown in SEQ ID NO: 7 of Example 3.

The sequence information of the lasso Spy resulting from topological modification was as shown below. The underlined parts were the sequences as same as those of the wild-type Spy (specifically, the amino acid sequences at positions 43 to 101, at positions 14 to 42, and at positions 1 to 13 of the wild-type Spy, respectively), and the bold parts were the introduced sequences of the newly-generated loop regions as compared to the wild-type Spy, which primarily included a sequence (GT/EL/VE/AS/RS) translated by the DNA endonuclease involved in molecular cloning, a TEV protease cleavage site (ENLYFQG) for proving the topological structure, an affinity purification tag (HHHHHH), and a flexible loop region consisting of G and S.

(SEQ ID NO: 11)
MKGSSHHHHHHVEASSGKTISTWISDGQVKDFYLYPGKYTFVETA
APDGYEVATAITFTVNEQGQVTVNGKATKGGSGGSGGSASGEDSA
THIKESKRDEDGKELAGATMELRDRSGGSGGSENLYFQGGSGGSA
HIVMVDAYKPTKGGSGGSGG

As shown in FIG. 17, the original isopeptide bond connectivity could be removed by mutating the active site Asp (aspartic acid, D) for forming the isopeptide bond in the lasso Spy to Ala (alanine, A) to obtain a knot Spy.

The sequence information of the knot Spy was as shown below. The underlined parts were the sequences as same as those of the wild-type Spy (specifically, the amino acid sequences at positions 43 to 101, at positions 14 to 42, and at positions 1 to 6 and 8 to 13 of the wild-type Spy, respectively); the shadow was the mutated active site at position 7; and the bold parts were the introduced sequences of the newly-generated loop regions as compared to the wild-type Spy, which primarily included a sequence (GT/EL/VE/AS/RS) translated by the DNA endonuclease involved in molecular cloning, a TEV protease cleavage site (ENLYFQG) for proving the topological structure, an affinity purification tag (HHHHHH), and a flexible loop region consisting of G and S.

(SEQ ID NO: 12)
MKGSSHHHHHHVEASSGKTISTWISDGQVKDFYLYPGKYTFVETAAPDGYEVAT
AITFTVNEQGQVTVNGKATKGGSGGSGGSASGEDSATHIKFSKRDEDGKELAGATME

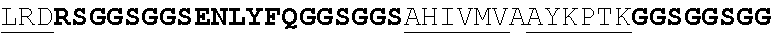

The lasso Spy and knot Spy were expressed and purified, and their basic physical and chemical properties were characterized.

The molecular weights of the two products were confirmed by high-performance liquid chromatography-electrospray mass spectrometry (LC-MS). The mass spectrometry conditions and operation methods in this experiment were as follows: the sample concentration was 0.5 mg/mL, the Acquity UPLC H-Class system and the Acquity QDa mass spectrometry detector were used for characterization, and the MassLynx V4.1 analysis software was used for integration.

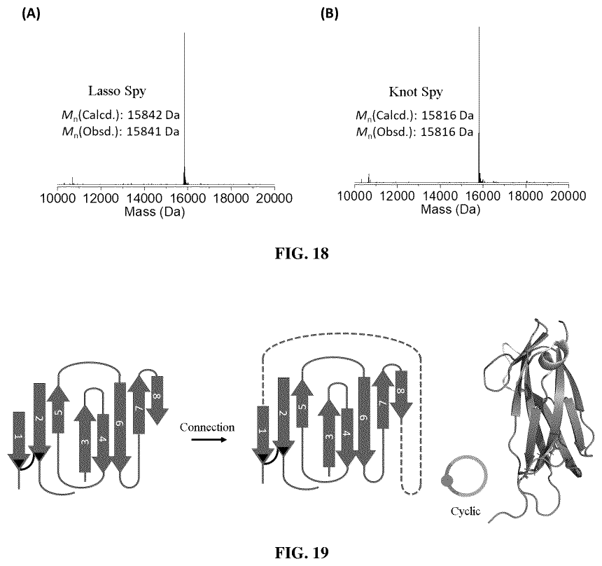

Experimental results: the results showed that the synthesized proteins met the requirement for the molecular weights of the target lasso structure and the knot structure (as shown in (A) and (B) of FIG. 18).

### Example 5: Design of Single-Domain Cyclic Spy and Linear Spy

The wild-type Spy was derived from the CnaB2 domain. Its crystallographic structure (PDB No.: 4MLI) and topology diagram were as shown in FIG. 12. Its crystallographic structure contained SpyTag and SpyCatcher. The Asp on the SpyTag could undergo an autocatalytic reaction with the Lys on the SpyCatcher to form a natural isopeptide bond that joined the two parts together. The isopeptide bond was regarded as an effectively connected loop region in the design of the present example.

Following the design idea of the design method for topological proteins provided in the present disclosure, the cyclic Spy was designed as follows: the wild-type Spy structure was analyzed and found to contain eight secondary structural motifs (named secondary structural motif 1, secondary structural motif 2 . . . secondary structural motif 7, and secondary structural motif 8, respectively) and eight loop regions (including the virtual loop region connecting the N-terminus and the C-terminus). According to the approach of splitting one loop region of the cyclic protein provided in step i) of the programmatic design method for the topological protein provided in the present disclosure, the loop region in the wild-type Spy was rationally split. There were a total of 8 splitting approaches, and each splitting approach corresponded to only one new connection approach, so there were eventually 8 different connection approaches, accordingly. Thereafter, each of the connection approaches was evaluated based on the evaluation method in step ii), and the structures with high scores were selected for design.

FIG. 19 showed the rewiring approach with the highest score among reconnections after splitting one loop region of the wild-type Spy. Specifically, FIG. 19 showed the rewiring between the secondary structural motif 1 and the secondary structural motif 8 of Spy.

Subsequently, according to the operations of step iii) in the programmatic design method for the topological protein provided in the present disclosure, a virtual loop region was generated at the above two positions to be connected, and all combinations of the generation orders were exhausted. The topological structures were determined for the resulting structures and the formation probability of the target topological structure was calculated. The optimal design approach of the cyclic Spy was finally screened out (with the formation probability of 100%), as shown in FIG. 19.

Furthermore, the following operations were carried out according to step iv) of the programmatic design method for the topological protein provided in the present disclosure: based on the length ranges of the newly-generated actual loop regions, a combination of lengths of the loop regions that were more aligned with the basis for scoring and evaluation disclosed in the specification of the present disclosure was selected as the specific number of the amino acids in each of the actual loop regions, and the amino acid sequences of the actual loop regions were thereby designed, whereby the sequence included a combination of an enzyme cleavage site, an affinity purification tag, a residual motif after a coupling reaction, and linking sequences consisting of glycine G and serine S. The topological structure and the corresponding secondary structural motif numberings of the cyclic Spy were as shown in FIG. 19.

The amino acid sequence of the wild-type Spy was as shown in SEQ ID NO: 7 of Example 3.

The sequence information of the cyclic Spy resulting from topological modification was as shown below. The underlined parts were the sequences as same as those of the wild-type Spy (specifically, the amino acid sequences at positions 14 to 42, at positions 43 to 101, and at positions 1 to 13 of the wild-type Spy, respectively), and the bold parts were the introduced sequences of the newly-generated loop regions as compared to the wild-type Spy, which primarily included a sequence (GT/KL/AS) translated by the DNA endonuclease involved in molecular cloning, a TEV protease cleavage site (ENLYFQG) for proving the topological structure, an affinity purification tag (HHHHHH), and a flexible loop region consisting of G and S.

(SEQ ID NO: 13)
MKSHHHHHHKLGGSGGSENLYFQGGTGSGGSGEDSATHIKFSKRD
EDGKELAGATMELRDGSASSGKTISTWISDGQVKDFYLYPGKYTF
VETAAPDGYEVATAITFTVNEQGQVTVNGKATKGGSGGSGGSGSA
HIVMVDAYKPTKGGSGGSGG

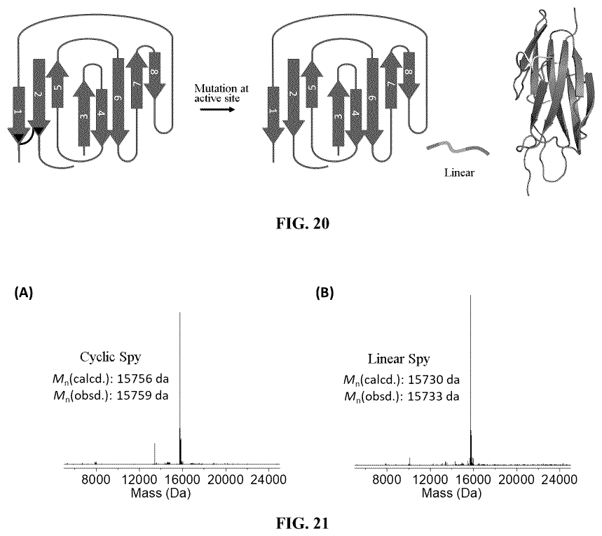

As shown in FIG. 20, the original isopeptide bond connectivity could be removed by mutating the active site Asp (aspartic acid, D) for forming the isopeptide bond in the cyclic Spy to Ala (alanine, A) to obtain a linear Spy.

The sequence information of the linear Spy was as shown below. The underlined parts were the sequences as same as those of the wild-type Spy (specifically, the amino acid sequences at positions 14 to 42, at positions 43 to 101, and at positions 1 to 6 and 8 to 13,respectively); the shadow was the mutated active site at position 7: and the bold parts were the introduced sequences of the newly-generated loop regions as compared to the wild-type Spy, which primarily included a sequence (GT/KL/AS) translated by the DNA endonuclease involved in molecular cloning, a TEV protease cleavage site (ENLYFQG) for proving the topological structure, an affinity purification tag (HHHHHH), and a flexible loop region consisting of G and S.

(SEQ ID NO: 14)
MKSHHHHHHKLGGSGGSENLYFQGGTGSGGSGEDSATHIKESKRDEDGKELA
GATMELRDGSASSGKTISTWISDGQVKDFYLYPGKYTFVETAAPDGYEVATAITFTVNE

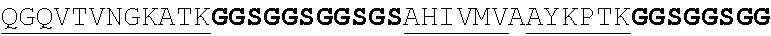

The cyclic Spy and linear Spy were expressed and purified, and their basic physical and chemical properties were characterized.

The molecular weights of the two products were confirmed by high-performance liquid chromatography-electrospray mass spectrometry (LC-MS). The mass spectrometry conditions and operation methods in this experiment were as follows: the sample concentration was 0.5 mg/mL, the Acquity UPLC H-Class system and the Acquity QDa mass spectrometry detector were used for characterization, and the MassLynx V4.1 analysis software was used for integration.

Experimental results: the results showed that the synthesized proteins met the requirement for the molecular weights of the target cyclic structure and the linear structure (as shown in (A) and (B) of FIG. 21, respectively).

What is claimed is:

1. A programmatic design method for a topological protein, the method comprising the following steps:
i) splitting an original structure of a protein-of-interest, and designing possible rewiring approaches according to a target topological structure:
ii) evaluating connection approaches between structural motifs and determining priorities of the connection approaches in a subsequent design:
iii) for each of the connection approaches, generating new virtual loop regions successively, exhausting all possible combinations of generation orders of the loop regions, creating corresponding spatial relationships, determining the chemical topology of the formed structures, calculating a formation probability of the target topological structure, and determining a length range of a newly-generated loop region; and
iv) designing a length and sequence of the newly-generated loop region of the topological protein.

2. The method according to claim 1, wherein specific operations of the steps i) to iv) are as follows:
i) splitting an original tertiary structure of the protein-of-interest by using a secondary structure as a motif, successively increasing the number of the loop regions to be split starting from two loop regions, giving priority to an approach where fewer loop regions will be split, determining a splitting approach, and then designing all possible new connection approaches between secondary structural motifs of the protein based on the target chemical topological structure:
ii) scoring and evaluating the new connection approaches designed in step i), and determining the priorities of different connection approaches in the subsequent design of a topological structure based on the results of scoring and evaluation:
iii) further designing the spatial relationship of each of the connection approaches in order of priorities based on the results of the scoring and evaluation in step ii), generating the corresponding spatial relationship by successively generating the new virtual loop region at each of positions to be connected and exhausting all possible combinations of the generation orders of the loop regions, determining the chemical topological structures of the protein obtained after generating the new virtual loop regions, and calculating the formation probability of the target topological structure in said connection approach, thereby determining a relative spatial relationship and the length range of the virtual loop region corresponding to the formation of the target topological structure in said connection approach; and
iv) preferably selecting specific lengths of actual loop regions based on the relative spatial relationship and the length range of the virtual loop region determined in step iii), and further designing the amino acid sequences of the actual loop regions as the sequences of the newly-generated loop regions of the topological protein to obtain a final topological protein.

3. The method according to claim 2, wherein the original tertiary structure of the protein-of-interest in step i) contains N secondary structural motifs and N loop regions, wherein the loop regions comprise the virtual loop region between N-terminus and C-terminus, and when the topological structure of a designed protein-of-interest is [2] catenane, the original tertiary structure of the protein-of-interest is split by the following method and a new connection approach is determined:
(1) splitting two loop regions in the original tertiary structure of the protein-of-interest, with a total of N(N-1)/2 splitting approaches and N(N-1)/2 new connection approaches;
(2) splitting three loop regions in the original tertiary structure of the protein-of-interest, with a total of N(N-1) (N-2)/6 splitting approaches and N(N-1) (N-2)/2 new connection approaches: or
(3) splitting M loop regions in the original tertiary structure of the protein-of-interest, with a total of N!/[(N-M)!×M!] splitting approaches and
$$\frac{N !}{( N - M ) ! \times M !} \times {\sum }_{L = 1}^{M - 1} ( M ! / 2 L ( M - L ) )$$
new connection approaches, wherein M is 4 or an integer greater than 4, and L is a positive integer from 1 to M-1:
preferably, performing subsequent evaluations and designs successively in order from the smallest to the largest number of the loop regions required to be rewired.

4. The method according to claim 2, wherein a basis for the scoring and evaluation in step ii) is as follows:
setting two evaluation criteria for each of the positions to be connected, i.e., (a) a Euclidean distance between the secondary structural motifs to be connected, and (b) a probability that the loop regions generated between the to-be-connected secondary structural motifs conform to the statistical law of the loop regions of all natural proteins:
calculating the probability of generating the new loop regions at the positions to be connected based on the evaluation criteria (a) and (b); and
calculating an overall generation probability taking account of all loop regions required to be regenerated in a current connection approach, scoring and ranking all connection approaches based on the probability, and selecting superior scoring groups for subsequent successive design.

5. The method according to claim 4, wherein specific operations of the scoring and evaluation are as follows:
(a1) counting the Euclidean distances of all loop regions in Protein Data Bank (PDB), calculating a ratio of the number of the loop regions corresponding to each of the Euclidean distances to the total number of the loop regions, and taking the ratio as the probability p1 of generating the loop regions at said Euclidean distance:
(b1) counting the Euclidean distance between the loop regions and the lengths of the loop regions of all proteins in PDB to obtain probability distribution of the lengths of the loop regions at a specific Euclidean distance, generating a virtual loop region at a target position by a minimum solvent accessible path as a minimum length of an actually generable loop region, performing an integral calculation on the probability distribution by taking said length as a lower limit of integration and taking the longest loop region counted under the current Euclidean distance as an upper limit of integration, to obtain a probability p2 that the actually generated loop regions conform to the law:
(c1) taking a product of the probabilities calculated in (a1) and (b1) as the probability p of actually generating the loop regions at the current position, i.e., p=p1×p2: and
(d1) taking a product of the probabilities of generating the loop regions calculated at all positions to be connected in the current connection approach as the probability pₜₒₜₐₗ, i.e., pₜₒₜₐₗ=Πpᵢ, of said connection approach, wherein said i is the number of all new loop regions required to be generated, performing the scoring and ranking based on the probability, and determining the priorities in the subsequent designs of the topological structures based on the ranking.

6. The method according to claim 2, wherein the step iii) comprises: generating new virtual loop regions between the secondary structural motifs by a minimum solvent accessible path in specific connection approaches: generating more than one new virtual loop region in each of the connection approaches, and exhausting all possible spatial relationships between newly-generated virtual loop regions by exhausting all combinations of the generation orders of the virtual loop regions, wherein the total number of all possible spatial relationships is n:
determining the topological structure of the designed protein corresponding to the newly-generated virtual loop regions in each of the generation order by calculating the Gauss linking number or the knot invariant, to obtain the number m of the topological structures that match the target topological structure;
calculating the probability m/n of generating the target topological structure in the current connection approach based on said n and m, and outputting the probability as the formation probability of the target topological structure in a current connection relationship; and
determining the length ranges of the actual loop regions based on the connection relationship and the spatial relationship that enable formation of the target topological structure, and imposing a length limitation based on the relative spatial relationships of the actual loop regions, and defining the length range of each of the loop regions that are adjacent to and cross each other based on the requirement that the difference between the length of the loop region relatively far away from a hydrophobic core of the folded protein and the length of the loop region relatively close to the hydrophobic core of the folded protein is not less than the length difference in the lower limits thereof, in order to maintain the relatively spatial relationships unchanged.

7. The method according to claim 2, wherein the step iv) comprises: selecting, based on the length ranges of the actual loop regions determined in step iii), combinations of the lengths of the loop regions that are more aligned with a basis for scoring and evaluation as the specific number of the amino acids in each of the actual loop regions, and designing amino acid sequences of the actual loop regions:
wherein the basis for the scoring and evaluation is as follows:
setting two evaluation criteria for each of the positions to be connected, i.e., (a) a Euclidean distance between the secondary structural motifs to be connected, and (b) a probability that the loop regions generated between the to-be-connected secondary structural motifs conform to the statistical law of the loop regions of all natural proteins:
calculating the probability of generating the new loop regions at the positions to be connected based on the evaluation criteria (a) and (b); and
calculating an overall generation probability taking account of all loop regions required to be regenerated in a current connection approach, scoring and ranking all connection approaches based on the probability, and selecting superior scoring groups for subsequent successive design:
preferably, the amino acid sequences of the actual loop regions are designed by any one of the following three methods: (1) directly designing a flexible linking loop region with the target number of amino acids, wherein the flexible linking loop region comprises any one of an enzyme cleavage site, an affinity purification tag, a residual motif after a coupling reaction, part or full sequence of the original loop region, or linking sequences consisting of glycine G and serine S, or any combination thereof: (2) searching structures similar to the two termini of the secondary structural motifs to be connected in PDB by a similar structural motif search algorithm, and selecting the loop regions with the lengths that meet the requirement as the loop region to be designed: and (3) designing linking loop regions with target lengths by a computer-assisted means:
more preferably, the enzyme cleavage site is any one of a Tobacco Etch Virus protease cleavage site (ENLYFQG), a Tobacco Vein Mottling Virus protease cleavage site (ETVRFQG), an enterokinase cleavage site (DDDDK), a coagulation factor Xa protease cleavage site (IDGR) or a WELQut protease cleavage site (WELQ): the affinity purification tag is any one of Histag (HHHHHH), Strep-Tag II (WSHPQFEK) or a Flag tag (DYKDDDDK); the residual amino acid sequence after the coupling reaction is any one of CFN, ESGSGK, LPETG or NHV; the similar structural motif search algorithm is any one of MASTER, FragBag or TOPOFIT; and the computer-assisted means is any one of a Rosetta loop modelling method, a SCUBA method, or a FoldX LoopReconstruction method.

8. The method according to claim 1, wherein the chemical topological structure of the topological protein is any one selected from the group consisting of a branched structure, a multicyclic structure, a knot structure, and a link structure, or any combination thereof;
preferably, the topological protein is a protein catenane having two or more mechanically-interlocked cyclic structures or a knot protein having a trefoil knot, 4₁ knot, 5₁ knot or 5₂ knot structure.

9. A topological protein, wherein the topological protein is designed by the method according to claim 1.

10. The topological protein according to claim 9, wherein the chemical topological structure of the protein is any one selected from the group consisting of a branched structure, a multicyclic structure, a knot structure, and a link structure, or any combination thereof; preferably, the topological protein is a protein catenane having two or more mechanically-interlocked cyclic structures or a knot protein having a trefoil knot, 4₁ knot, 5₁ knot or 5₂ knot structure.
